(19) United States
(12) Patent Application Publication
(10) Pub. No.: US 2016/0375220 A1
(43) Pub. Date: Dec. 29, 2016

(54) METHOD TO MAINTAIN PEACE THROUGH ELECTROMAGNETIC ENERGY TARGETED TO THE BRAIN

(71) Applicant: Joel Steven Goldberg, Hillsborough, NC (US)

(72) Inventor: Joel Steven Goldberg, Hillsborough, NC (US)

(21) Appl. No.: 15/057,146

(22) Filed: Mar. 1, 2016

(51) Int. Cl. A61M 21/02; A61N 2/02; A61N 2/00

(52) U.S. Cl. CPC A61M 21/02; A61N 2/006; A61N 2/02; A61M 2021/0055

(57) ABSTRACT

World peace and resolution of conflicts depends upon methods to deter use of weapons of mass destruction. Negotiations are the primary method to resolve conflicts. Pain, especially non-tissue sources of pain, is ubiquitous in the minds of humans and is an impediment to successful conflict resolution. It is proposed that leaders who are at peace with themselves are more likely to negotiate win-win resolutions of conflicts. When negotiations fail and deployment of weapons of mass destruction by a country is imminent, targeting the brain of its leaders to change their intentions may be reasonable and necessary. Concentrated extremely low frequency (ELF) electromagnetic waves can penetrate the cranium in the most conductive portion of the skull, the pterion-temporal lobe intersection, and produce effects in the anterior temporal lobe that contains the amygdala. Remote stimulation of the temporal lobe may cause lethal or incapacitating seizures. Stimulation of the amygdala produces fear in a high percentage of humans and in some instances respiratory depression. Using the Belousov-Zhabotinsky (B-Z) reaction as a model of brain activity, an early prototype of this weapon was developed. In order to prevent another arms race this technology should be assigned to the United Nations.

### CROSS-REFERENCES TO RELATED APPLICATIONS

None

### FEDERALLY FUNDED RESEARCH

Not applicable

### BACKGROUND OF THE INVENTION

The most serious man-made threat to civilization is the deployment of weapons of mass destruction by a leader or group of leaders who are impulsive, immature and fearless. If such weapons are launched on a society that is not able to defend itself with antiballistic missiles a catastrophe is inevitable. A catastrophe will not only occur from those who experience a direct hit, but such an event is predicted to produce global traumatic stress. Such stress has the potential to cripple the minds of many inhabitants on earth. (Goldberg, 2008)

Throughout history resolution of conflict by force has evolved from hand to hand contact to use of projectile weapons. Weapons from wave energy are uniquely different than present weapons. Interference between waves is not limited by the confines of space. Unlike weapons made of matter, weapons composed of waves can coexist in the brain and interfere with brain electrophysiology. These weapons can be remotely fired and penetrate the cranium and brain. It is the purpose of this invention to describe a method to alter the mind(s) of a leader or leaders by focusing electromagnetic waves into the brain of these leaders to cripple their ability to perform heinous acts on society. Such a weapon could be lethal or incapacitating. In order to avoid another arms race, this weapon should be authorized to be used by the United Nations.

The anatomy of the skull predicts where electromagnetic waves are most likely to penetrate. Because the bones of the cranium are thinnest in the region of the pterion and more specifically where the pterion is contiguous with the temporal bone, an electromagnetic wave is most likely to effectively penetrate this area. (FIG. 1) The average thickness of the skull is approximately 6.5 mm but the thickness at the pterion-temporal bone intersection is 1 mm. (Ma, Baillie, & Stringer, 2012) The relative impedance of bone is 80 times that of other tissues. (Gabriel, Lau, & Gabriel, 1996) Extremely low frequency (ELF) electromagnetic waves of 5-40 Hz are expected to penetrate the cranium and brain and alter the electrophysiology of the temporal lobe through wave interference or energy transfer since both electric and magnetic fields are sources of energy. Deeper stimulation of the amygdala is also likely. With geo-positioning technology electromagnetic waves can be precisely targeted.

Changing the trajectory of chaotic brain activity may require only small modifications of brain electrical activity to change associated behaviors. Stimulation of the amygdala within the anterior temporal lobe has reliably produced fear. (Gloor, Olivier, Quesney, Andermann, & Horowitz, 1982) Respiratory depression has also been reported from amygdala stimulation. (Dlouhy et al., 2015) If stimulation of the temporal lobe produces seizures, then incapacitation or death may be likely.

Previous work has shown that frequencies between 5-40 Hz stimulate cortical neurons as measured by phosphene activity. (Goldberg, 2016) The frequency of phosphene activity is much less than that of the frequency of stimulation, suggesting that the phosphene frequency is a beat frequency generated by the superposition of external electromagnetic waves with natural brain wave frequencies.

The International Association for the Study of Pain defines pain as an unpleasant sensory and emotional experience associated with actual or potential tissue damage or described in terms of such damage. This definition is incomplete since it considers pain as a cause of actual or potential tissue damage. A more complete definition of pain is that which causes suffering and/or distress.

Pain and peace are states within the mind that are not complementary. If resolution of conflicts between leaders and their countries were easy with win-win outcomes, then the world would be a more peaceful place. Unfortunately this is not always the case and leaders bring to the negotiation table their pain from many sources. In fact negotiations often become competitive rather than cooperative and end with compromise where parties agree to accept something less then they originally wanted. (Coleman 2015) It is proposed that if the minds of world leaders were at peace that war would be a less likely choice for resolution of conflicts and more negotiations would end with win-win resolutions.

Within the mind three causes of pain may exist: 1) feelings including emotions 2) memories and 3) thoughts. Feelings can be site specific causes of pain that may be transmitted by the afferent or sympathetic nervous system. Emotions such as anger, fear, grief, envy and guilt are a subset of painful feelings which exist within the mind and may relate to neuroendocrine function. Memories are images within the mind that cause pain because of intrusiveness and/or content. They may be described as flashbacks that are too painful to forget. Thoughts, the language within the mind, may be intrusive causing pain and interfering with concentration. Thoughts may have evil, contemptuous, and blasphemous content. When these causes of pain exist in the mind there is uneasiness, a discomforting difference, or conflict. World leaders need to identify and ameliorate sources of their conflict so that decisions can come from rational thought unencumbered by a mind filled with painful feelings, memories and thoughts. To better understand how pain in the mind is far more than nociception some examples follow:

Since most decisions in life and nearly all political decisions have an economic consideration it is not surprising that economic matters are a source of pain. A few of these will be addressed:

### Economic Sources of Pain

1. Inflation, an economic policy adopted by nearly every country and supported by its leaders causes pain because the purchasing power of a currency constantly erodes and constituents cannot prepare for their financial future without assuming risk of principal.
2. Debt, both government and personal, produces insidious pain that in many countries and families is passed on to future generations.
3. Leaders who are insensitive to poverty and starvation ignore a well-recognized source of pain that may be ameliorated by education and redistribution of wealth.
4. Excessive taxation and tariffs proposed by leaders produce pain by siphoning off the earnings of constituents.
5. Threats to employment, income or property are familiar sources of pain.

### Political Sources of Pain

The world is divided into 195 sovereign countries and it is unlikely that a single political system will emerge from this diversity. Leaders of governments can inflict pain on constituents and societies in the following ways:
1. Not providing safety and security for its constituents
2. Not allowing freedom of passage
3. Conscription
4. Unjust laws
5. Government intrusion on individual privacy

### Religious Ideologies as Sources of Pain

Religious ideologies may be difficult to change because they are faith based:
1. Claiming and acting as if one religion is superior to all others
2. Coerced conversion
3. Discrimination in which laws favor a religious sect.

Economic, political and religious sources of pain are only examples of pain experienced by leaders and inflicted upon societies. Other sources of pain include discrimination, ethnic cleansing and genocide, harm to others either physical or through communication. Also pain from an individual's past experiences and pain from interpersonal relationships are nearly ubiquitous in the minds of humans. Pain from natural disasters such as floods, hurricanes, earthquakes, typhoons and tornados are beyond human control. This list of pain that exist in the minds of humans is incomplete, but it is emphasized so that the reader understands that not all pain relates to tissue destruction. Although not proven, it seems plausible that win-win resolutions of conflicts may be more likely if leaders were as pain free as possible when they enter into negotiations.

When leaders cannot resolve conflict and there is a threat of war from weapons of mass destruction, attempts to change the mind of a leader or leaders using targeted electromagnetic energy may be necessary and reasonable. The outcome of remotely targeted electromagnetic waves to the temporal lobe is unknown but some predictions can be made. Targeting electromagnetic waves to the temporal lobe could be lethal by causing status epilepticus, sudden unexpected death in epilepsy (SUDEP) or asphyxiation from loss of airway. Other potential outcomes of temporal lobe stimulation include fear from stimulation of the amygdala and less commonly respiratory depression. This invention describes how such a technology can be developed.

### Prior Art

| Pat. | Working | Targeted | Electromagnetic | Specified range of |
|---|---|---|---|---|
| No. | prototype | brain site | fields | frequency |
| U.S. Pat. No. 3,393,279 | No | No | Yes | 20 kHz-200 kHz |
| U.S. Pat. No. 3,951,134 | No | No | Yes | 100 & 210 MHz |
| U.S. Pat. No. 6,488,617 | No | No | Magnetic | No |
| U.S. Pat. No. 6,292,688 | Yes | Auditory | Yes | 8, 13, 16, 18, 26 Hz |
| | | and visual | | |
| U.S. Pat. No. 6,506,148 | Yes | No | Yes | 0.1-15 Hz |
| U.S. Pat. No. | No | Yes | Magnetic | Maximum 0.3 Hz |
| 5,707,334A | | amygdala | | |
| U.S. Pat. No. | No | No | Yes | No |
| 7,051,636B1 | | | | |
| This | Yes | Yes | Yes | Yes ELF 5-40 Hz |
| invention | | | | |

### DESCRIPTION OF THE DRAWINGS

FIG. 1 shows a human skull. Label 1 is the pterion. Label 2 is the intersection of the pterion with the temporal bone, the most probable location of thinnest bone.

FIG. 2 shows the location of the temporal lobe within the cranium. Label 1 shows the anterior portion of the temporal lobe.

FIG. 3 shows the temporal lobe. Label 1 is the amygdala within the anterior temporal lobe.

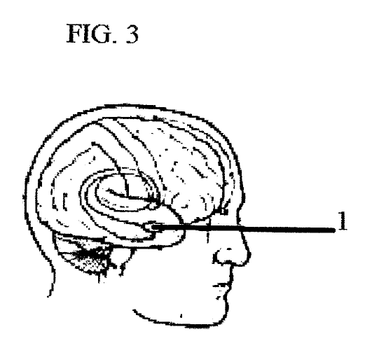

FIG. 4 shows the apparatus used to demonstrate wave interference of the B-Z reaction. Label 1 is a platinum electrode. Label 2 is a graphite electrode. Labels 3 and 4 are gold electrodes. Label 5 are the reagents of the B-Z reaction.

FIG. 5 is an oscilloscope tracing of a B-Z reaction demonstrating wave interference produce by electromagnetic waves from a function generator attached to gold electrodes.

### DETAILED DESCRIPTION OF THE INVENTION

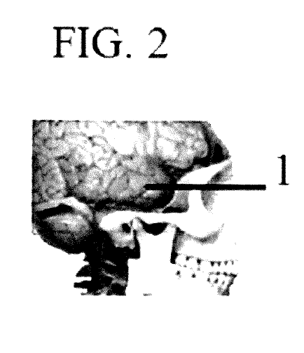

Targeting electromagnetic waves to the brain will require a source of electromagnetic waves, concentration of the waves, targeting of the waves and measurement of outcome. Many devices exist as sources of frequency dependent electromagnetic waves that can originate from a satellite in space or originate on earth directly or as a result of reflection from the ionosphere as demonstrated in the High Frequency Active Auroral Research (HAARP) experiments. The frequencies of waves most likely to interfere with the natural frequency of the brain are in the range of 5-40 Hz and square waves or its equivalent. (Goldberg, 2016) Since the least insulated area of brain is under the thinnest portion of the cranium, the pterion-temporal bone intersection, the most likely target is the anterior temporal lobe. (FIGS. 2 & 3) Stimulation of the temporal lobe with extremely low frequency (ELF) electromagnetic waves in this range can produce beat frequencies that are detectable. In humans, amygdala stimulation has been reliably associated with generation of fear which may be a deterrent for actions. (Gloor, et al., 1982) In some instances stimulation of the amygdala produces respiratory depression.

Electromagnetic waves targeted at the brain may produce changes in brain activity by direct interaction with neurons, energy transfer from the electric or magnetic field densities, or through interference of brain wave activity. Harnessing this energy for the purpose of altering the mind is possible.

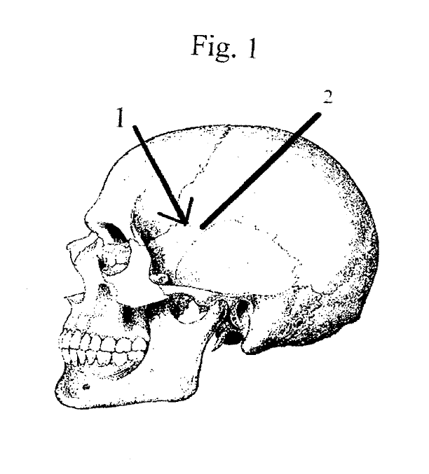

Previously it has been shown that a changing electromagnetic field can alter the energy from a chaotic, autocatalytic, oscillating chemical reaction which is a model of brain activity. (Goldberg, 2015) It has also been previously shown that the scalp over the pterions and more likely over the pterion-temporal lobe intersection has the least impedance when electrodes are attached. (Goldberg, 2016) In most races the pterions overlie the anterior temporal lobes beneath which lies the amygdala. (FIGS. 1, 2, & 3) Stimulation of amygdala produces fear that may affect behavior. (Gloor, et al., 1982)

This invention describes a remote system to alter the normal brain electrophysiology of a leader or group of leaders who have intentions to produce heinous crimes on society.

### Experimental Section

The Belousov-Zhabotinsky (B-Z) reaction is used as the model of brain activity. (Zhang, Gyorgyi, & Peltier, 1993) The reaction is autocatalytic, chaotic and produces electrical potentials similar to action potentials in the brain. The frequency of the reaction is variable but ranges from 1/60 to 1/10 Hz. The wave oscillations can be captured on an oscilloscope.

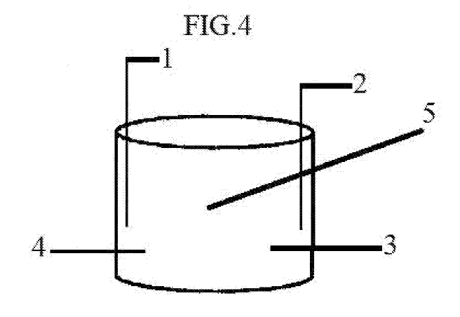

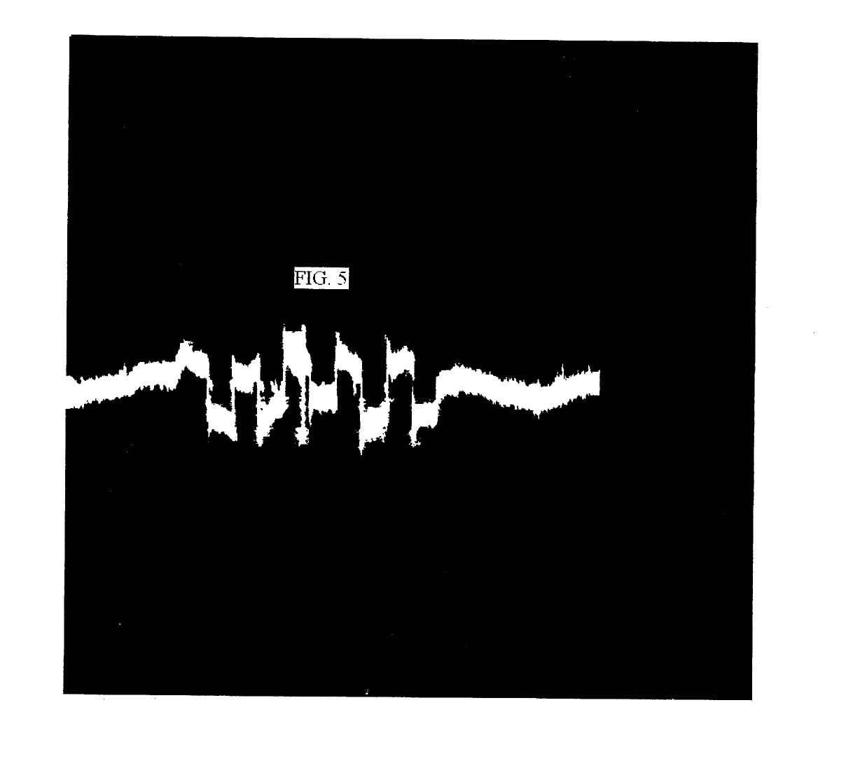

Electromagnetic square waves were generated from an arbitrary function generator. The probes carrying the electromagnetic waves were connected to gold electrodes that were inserted in the vessel containing the B-Z reaction. (FIG. 4) The frequency and amplitude of the electromagnetic square waves were adjusted until interference patterns were observed on the oscilloscope. (FIG. 5)

Interference patterns were dependent on output frequency and amplitude settings from the function generator. Output frequencies that approached the frequency of the B-Z reaction were shown to produce the most easily detected interference. (FIG. 5) Increasing amplitude of the generated signal also increased interference patterns.

### Benefits to Society

The most serious threat to world stability is the use of weapons of mass destruction by fearless aggressive evil leaders. When negotiations reach an impasse and use of such weapons is imminent, then actions are needed to deter the use of these weapons. Focused remote electromagnetic waves may be lethal, incapacitating or alter behavior of leaders and possibly their followers. Because the cranium is an impedance to cortical stimulation, electromagnetic waves targeted to the temporal lobe and the deeper the amygdala within the lobe are most likely to produce effects. The prototype of this invention demonstrates how the electrical potentials of an autocatalytic, chaotic, determinant chemical reaction (B-Z reaction) that models brain activity can be changed by electromagnetic waves. The source, specific frequencies, amplitude and waveform conformation that significantly alter brain electrophysiology need to be further studied. If such a defensive weapon is developed, it should be assigned to the United Nations.

### REFERENCES

Coleman, P. T., Deutsch, M. (2015). Morton Deutsch: A Pioner in Developing Peace Psychology: Springer International Publishing.
Dlouhy, B. J., Gehlbach, B. K., Kreple, C. J., Kawasaki, H., Oya, H., Buzza, C., et al. (2015). Breathing Inhibited When Seizures Spread to the Amygdala and upon Amygdala Stimulation. J Neurosci, 35(28), 10281-10289.
Gabriel, S., Lau, R. W., & Gabriel, C. (1996). The dielectric properties of biological tissues: II. Measurements in the frequency range 10 Hz to 20 GHz. Phys Med Biol, 41(11), 2251-2269.
Gloor, P., Olivier, A., Quesney, L. F., Andermann, F., & Horowitz, S. (1982). The role of the limbic system in experiential phenomena of temporal lobe epilepsy. Ann Neurol, 12(2), 129-144.
Goldberg, J. S. (2008). Global Traumatic Stress: Hypothetical Events and Possible Solutions. Traumatology, 20(10), 1-3.
Goldberg, J. S. (2015). Thermodynamic model of a nervous system US Patent No. 2015/0379898 A1
Goldberg, J. S. (2016). Method to optimize electrode placement for cranial electrical stimulation. U.S. patent application Ser. No. 14/995,376
Ma, S., Baillie, L. J., & Stringer, M. D. (2012). Reappraising the surface anatomy of the pterion and its relationship to the middle meningeal artery. Clin Anat, 25(3), 330-339.
Zhang, D., Gyorgyi, L., & Peltier, W. R. (1993). Deterministic chaos in the Belousov-Zhabotinsky reaction: Experiments and simulations. Chaos, 3(4), 723-745.

Having described my invention, I claim:

1. A method to produce behavioral changes in a human from remote transfer of electromagnetic energy to the temporal lobe of the brain.

2. The method of claim 1 where electromagnetic energy is transferred to the amygdala.

3. A method to produce a seizure in a human by remote transfer of electromagnetic energy to the temporal lobe of a human.
